(19) United States
(12) Patent Application Publication
(10) Pub. No.: US 2021/0059861 A1
(43) Pub. Date: Mar. 4, 2021

(54) METHOD AND TAPE FOR SCAR CARE

(71) Applicants: Spidertech Inc., Toronto (CA); Sunnybrook Research Institute, Toronto (CA)

(72) Inventors: Ray Arbesman, Toronto (CA); Marc Jeschke, Toronto (CA)

(21) Appl. No.: 16/555,311

(22) Filed: Aug. 29, 2019

(51) Int. Cl. A61F 13/02

(52) U.S. Cl. CPC A61F 13/025; A61L 15/58; A61F 13/024

(57) ABSTRACT

Disclosed herein is a tape, and related kits and methods, which is usable for scar care (e.g. for hypertrophic, keloid, or other scars). The tape may be used to treat, prevent, or inhibit scar pain, itching, and/or discomfort, and/or to improve or hasten scar healing, and/or to decrease scar height and redness, and/or to improve quality of life. The tape is highly stretchable along its width, but minimally stretchable or non-stretchable along its length. In use, the tape is placed over the scar, so that the length of the tape is aligned with the length of the scar, and stretched widthwise across the scar to pull the opposed sides of the scar towards each other.

### FIELD

This document relates to wound and scar care. More specifically, this document relates to methods and tapes for scar care, including care of hypertrophic scars, and keloid scars.

### BACKGROUND

United States Patent Application Publication No. US20170049629A1 (Arbesman et al.) discloses a wound care tape that includes a sterile high-stretch strip of woven material having a first face and an opposed second face. A sterile adhesive is on the first face in a discontinuous pattern. A set of spaced apart adhesive-covered portions of the first face are covered by the adhesive, and a set of spaced apart adhesive-free portions of the first face are free of the adhesive.

### SUMMARY

According to some aspects, a method for scar care includes a) providing a strip of woven material having i) a first face and an opposed second face, ii) a length and a width, wherein the width is shorter than the length, iii) a length stretch ratio along the length and a width stretch ratio along the width, wherein the width stretch ratio is greater than the length stretch ratio, and iv) a first side portion and a second side portion spaced widthwise from the first side portion. An adhesive is on the first face in a discontinuous pattern. The method further includes b) adhering the first side portion of the tape to a patient's skin on a first side of a scar; c) stretching the tape widthwise; and d) adhering the second side portion of the tape to the patient's skin on a second side of the scar.

In some examples, the tape further includes a central portion between the first side portion and the second side portion, and the first side portion includes a first plurality of fingers extending outwardly from the central portion, and the second side portion includes a second plurality of fingers extending outwardly from the first side portion.

In some examples, the method further includes e) adhering the central portion to the scar. In some examples, step e) is carried out before step c), and step c) is carried out before steps b) and d).

According to some aspects, a tape for scar care includes a strip of woven material having i) a first face and an opposed second face, ii) a length and a width, wherein the width is shorter than the length, iii) a length stretch ratio along the length and a width stretch ratio along the width, wherein the width stretch ratio is greater than the length stretch ratio, and iv) a longitudinally extending central portion, a first side portion on a first side of the central portion, wherein the first side portion comprises a first plurality of fingers extending outwardly from the central portion, and a second side portion on a second side of the central portion, wherein the second side portion comprises a second plurality of fingers extending outwardly from the central portion. An adhesive is on the first face in a discontinuous pattern.

In some examples, a release liner is on the second face of the tape. The release liner can be perforated along a boundary between the central portion and the first plurality of fingers and the along a boundary between the central portion and the second plurality of fingers.

### BRIEF DESCRIPTION OF THE DRAWINGS

FIG. 1 is a perspective view of an example tape for scar care;

FIG. 2 is an enlarged view of the region shown in circle 2 in FIG. 1;

FIG. 3 is a top view of the tape of FIG. 1;

FIG. 4 is a bottom view of the tape of FIG. 1;

FIG. 5A is a top view of the tape of FIG. 1, in an unstretched configuration;

FIG. 5B is a top view of the tape of FIG. 5A, in a stretched configuration;

FIG. 6 is a side view of a kit including the tape of FIG. 1 on a release liner;

FIG. 7 is a bottom view of the kit of FIG. 6;

FIG. 8 shows the tape of FIG. 1 in use, stretched widthwise across a scar and adhered to a user's skin;

FIG. 9 is a perspective view of an example roll of tape, showing how the tape of FIG. 1 may be cut therefrom in order to fabricate the precut tape; and

FIG. 10 is a top view of an alternative example tape;

FIG. 11 is a bottom view of the tape of FIG. 10; and

FIG. 12 shows the tape of FIG. 11 in use, stretched widthwise across a scar and adhered to a user's skin.

### DETAILED DESCRIPTION

Disclosed herein is a tape, and related kits and methods, which may be used for wound care and/or scar care (e.g. for hypertrophic, keloid, or other scars). For example, the tape may be used to treat, prevent, or inhibit scar pain, itching, and/or discomfort, and/or to improve or hasten scar healing.

As will be described in further detail below, the tape may be configured such that it is highly stretchable along its width, but minimally stretchable or non-stretchable along its length. In use, the tape may be placed over the scar, so that the length of the tape is aligned with the length of the scar, and stretched widthwise across the scar to pull the opposed sides of the scar towards each other. The stretchable nature of the tape can result in compression and can deliver continuous pressure on the scar.

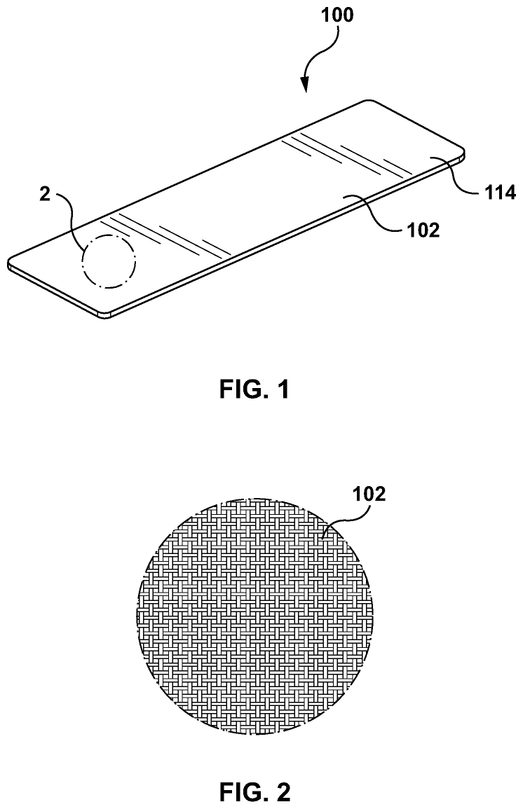

Referring now to FIG. 1, an example tape 100 is shown. The tape 100 includes a strip 102 of woven material. The woven nature of the strip 102 is shown schematically in FIG. 2. The woven material may be or may include, for example, woven cotton (similar to or the same as woven cotton used in kinesiology tapes).

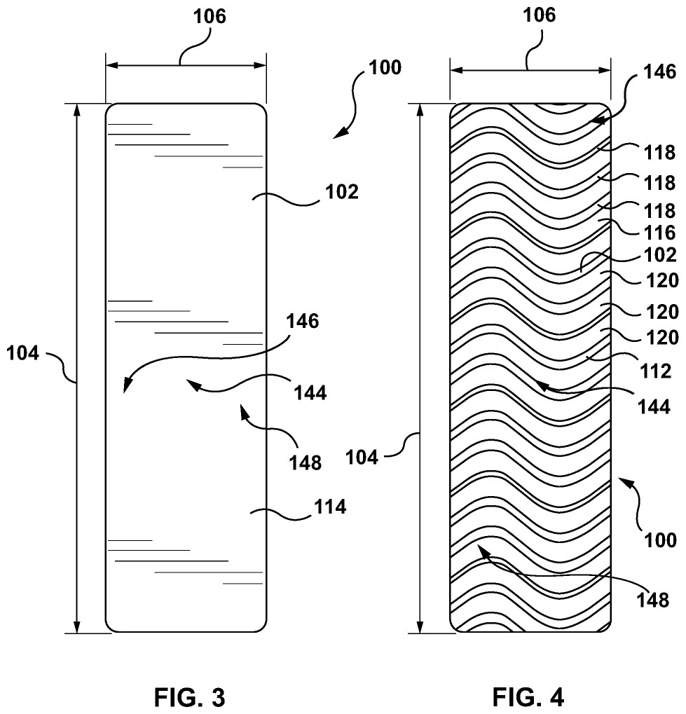

Referring to FIGS. 3 and 4, the strip 102 has a length 104 and a width 106 (wherein length 104 and width 106 refer to the dimensions when the tape 100 is in an unstretched or natural configuration). The width 106 is shorter than the length 104. For example, the length 104 may be at least about 2 times the width 106, or at least about 2.5 times the width 106, or at least about 2.75 times the width 106. In some examples, the length 104 may be between about 130 mm and 270 mm, and the width 106 may be between about 45 mm and 100 mm. In some examples, the length 104 may be between about 160 mm and about 240 mm, and the width 106 may be between about 55 mm and about 90 mm. In one particular example, the length 104 is about 178 mm, and the width 106 is about 60 mm. In another particular example, the length 104 is about 203 mm, and the width 106 is about 70 mm. In another example, the length 104 is about 216 mm, and the width 106 is about 80 mm.

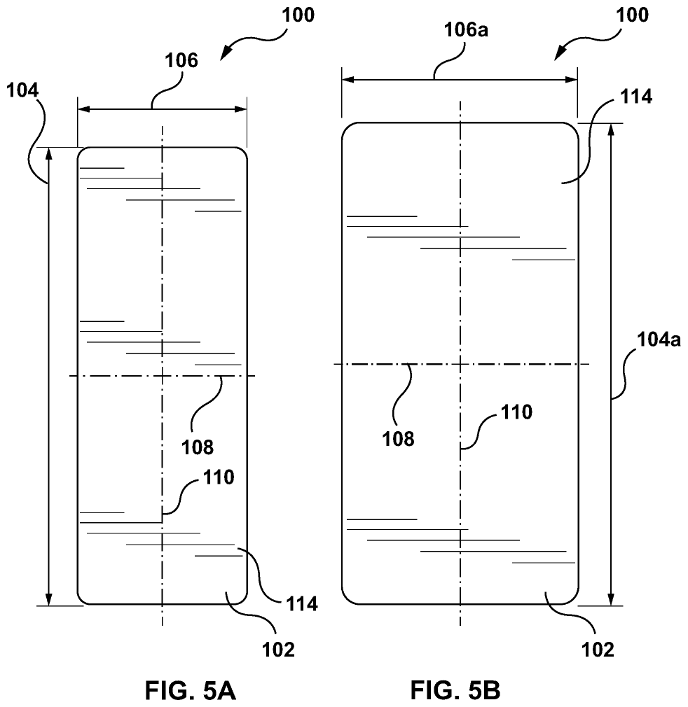

The strip 102 has a stretch ratio along its length 104 (also referred to as a length stretch ratio), and a stretch ratio along its width 106 (also referred to as a width stretch ratio). As used herein, the term ‘stretch ratio’ refers to a dimension of the strip 102 when stretched elastically to its maximum extent, divided by a dimension of the strip 102 when unstretched. For example, referring to FIG. 5A, the tape 100 is shown in an unstretched or natural configuration. Referring to FIG. 5B, the tape 100 is shown in a stretched configuration, wherein the strip 102 is stretched elastically to its maximum extent in both a widthwise and lengthwise direction (i.e. along transverse axis 108 and longitudinal axis 110). The width 106a of the strip 102 when the strip 102 is stretched elastically to its maximum extent along transverse axis 108, divided by its width 106 when unstretched, is its width stretch ratio. The length 104a of the tape when the tape is stretched elastically to its maximum extent along longitudinal axis 110, divided by its length 104 when unstretched, is its length stretch ratio.

In the example shown, the width stretch ratio is greater than the length stretch ratio. For example, the strip 102 may be at least twice as stretchable in a widthwise direction than in a lengthwise direction. For further example, the width stretch ratio may be at least 1.4 or about 1.4, and the length stretch ratio may be at most 1.3 or about 1.3. For further example, the width stretch ratio may be at least 1.6 or about 1.6, and the length stretch ratio may be at most 1.2 or about 1.2. For further example, the length stretch ratio may be between 1.4 and 2.0, and the width stretch ratio may be between 1.0 and 1.3.

In this document, a stretch ratio of 1.4 or greater may be referred to as a ‘high’ stretch ratio, and a stretch ratio of 1.3 or less (including a stretch ratio of 1) may be referred to as a ‘low’ stretch ratio. A material with a stretch ratio of 1 may also be referred to as ‘non-stretchable’. Furthermore, the transverse axis 108, along which the strip 102 has a greater stretch ratio, may be referred to as a ‘greater stretch axis’. The longitudinal axis 110, along which the strip 102 has a lower stretch ratio, may be referred to as a ‘lesser stretch axis.

Referring back to FIG. 3, the tape has a central portion 144 that extends longitudinally, a first side portion 146 on a first side of the central portion, and a second side portion 148 on a second side of the central portion.

Referring still to FIG. 3 and also to FIG. 4, the strip 102 has a first face 112 (shown in FIG. 4), and an opposed second face 114 (shown in FIG. 3). In use, the first face 112 faces the skin of the user, and the second face 114 faces away from the skin of the user.

An adhesive 116 is on the first face 112. Referring still to FIG. 4, in the example shown, the adhesive 116 is on the first face 112 in a discontinuous pattern. That is, the first face 112 includes a set of adhesive-covered portions (i.e. portions that are covered by the adhesive 116), and a set of adhesive-free portions 118 (i.e. portions that are free of adhesive 116, so that the first face is exposed). The adhesive-covered portions and adhesive-free portions 118 are provided in an alternating pattern, so that the adhesive-covered portions are spaced apart from each other, and the adhesive-free portions 118 are spaced apart from each other.

Referring still to FIG. 4, in the example shown, the adhesive 116 is provided on the adhesive covered portions in bands 120. The bands 120 extend non-linearly, and in the example shown are generally wave-shaped. The wave-shaped bands 120 extend generally widthwise across the tape, along the transverse axis 108 (i.e. along the greater stretch axis 108, shown in FIGS. 5A and 5B).

This discontinuous pattern of the adhesive 110 may allow for the tape 100 to accommodate movement, even when adhered to skin.

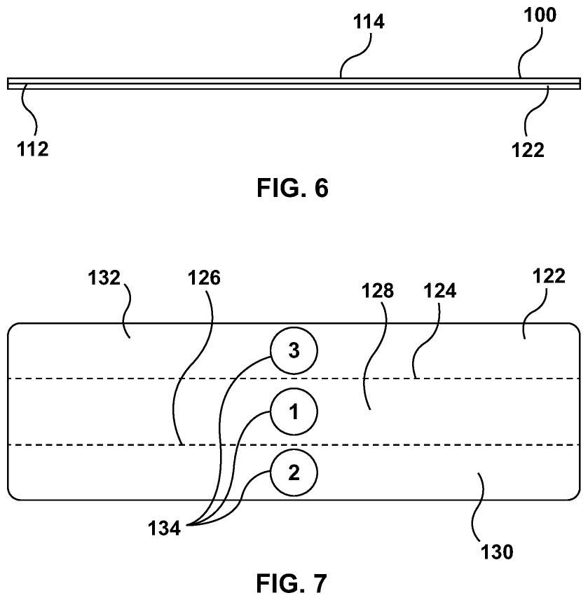

Referring to FIGS. 6 and 7, in the example shown, the tape 100 is provided in a kit with a release liner 122. The release liner 122 is adhered to the first face 112. The release liner 122 may in some examples include one or more lines of weakness, so that it may be severed along the lines of weakness and the portions thereof removed stepwise, in order to facilitate stepwise application of the tape 100. In the example shown, the release liner includes a first line of weakness 124 and a second line of weakness 126. In the example shown, each line of weakness 124, 126 is a perforated line. In alternative examples, the lines of weakness may be creases or score-lines.

Referring still to FIGS. 6 and 7, in the example shown, the first 124 and second 126 lines of weakness are spaced apart in a widthwise direction, along the transverse 108 axis 110 (i.e. greater stretch axis 108, shown in FIGS. 5A and 5B), and extend in a lengthwise direction, along the longitudinal axis 110 (i.e. lesser stretch axis 110, shown in FIGS. 5A and 5B). The first 124 and second 126 lines of weakness demarcate a central portion 128 of the release liner 122, and first 130 and second 132 side portions of the release liner 122. The central portion 128 of the release liner overlies the central portion 144 of the tape, and the first 130 and second 132 side portions of the release liner overlie the first 146 and second 148 side portions of the tape.

Referring still to FIG. 7, in the example shown, the release liner 122 further includes markings 134 to assist the user in the application of the tape 100. The markings as shown are numerical markings, indicating an order in which the central portion 128 and side portions 130, 132 of the release liner 122 may be removed, in order to facilitate stepwise application of the tape 100. In other examples, the markings may be alphabetical markings, or pictorial markings. In other examples, markings may be provided elsewhere, for example on the tape or on a separate instruction sheet.

The tape disclosed herein may in some examples be sterile. For example, the tape may be provided in a kit that includes a package having a sealed and sterile interior volume, with the tape and the release liner in the interior volume. The package may be a medical grade peel pouch. The package, with the tape and the release liner sealed in the interior volume, may in some examples be sterilized by steam sterilization. The package may be opened immediately prior to use of the tape. In alternative examples, a plurality of tapes may be provided in a single package.

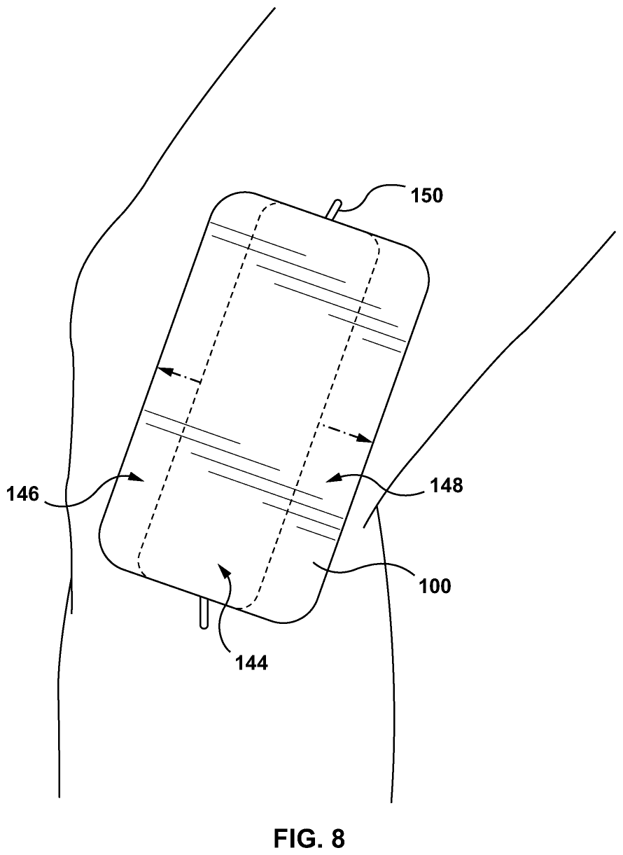

Referring now to FIG. 8, a method of treating a scar 150 will be described. The method will be described with respect to tape 100; however, the method may be carried out with other tapes, and tape 100 may be used according to other methods.

As a first step, the release liner may be removed from the tape. Optionally, the central portion 128 of the release liner may be removed from the tape 100 first, by tearing the release liner along lines 124 and 126. The tape 100 may be positioned adjacent the scar 150. The tape 100 may be positioned so that the first face 112 (shown in FIG. 4) faces the scar 150, and so that the longitudinal axis 110 (i.e. lesser stretch axis 110) is generally aligned along the scar 150, and the transverse axis 108 (i.e. lesser stretch axis 108) extends generally transverse to the scar 150. The central portion 144 of the tape 100 may then be adhered to the skin, directly over the scar 150.

The first side portion 130 of the release liner 122 may then be removed from the tape 100. The first side portion 146 of the tape may then be stretched away form the central portion 144, and adhered to the skin on a first side of the scar 150. Similarly, the second side portion 132 of the release liner 122 may then be removed from the tape 100. The second side portion 148 of the tape 100 may then be stretched away from the central portion 144, and adhered to the skin on a second side of the scar 150. The tape may then be rubbed to improve adhesion.

Due to the elastic nature of the tape, the opposed sides of the scar are pulled together by the tape, along the length of the scar. This can increase comfort, and decrease pain and itching. The tape can be left on the scar for a prolonged period of time, for example up to 7 days or up to 14 days. The tape can then be removed and replaced with a new tape. The stretchable nature of the tape is shown schematically in dotted lines in FIG. 8.

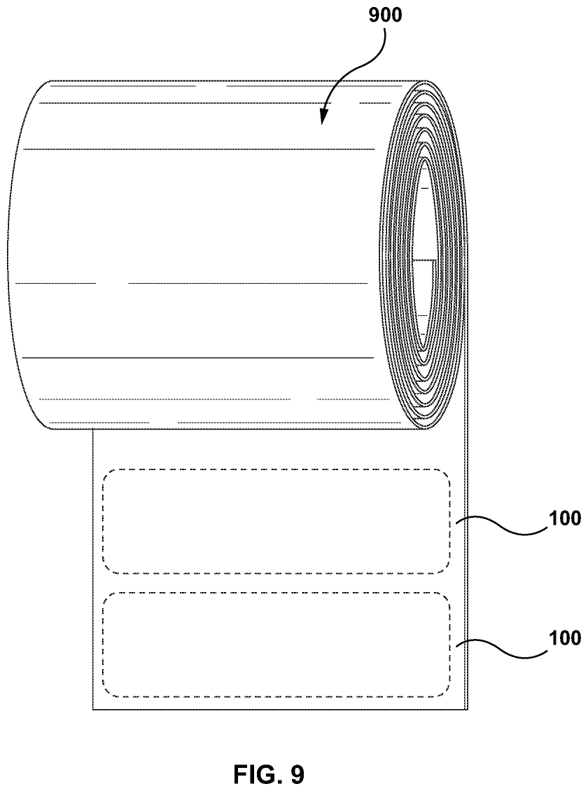

Referring to FIG. 9, a schematic diagram is shown illustrating how the tape 100 may be cut from a commercially available roll 900 of tape (e.g. kinesiology tape), in order to provide precut tapes. The roll 900 of tape may have a high stretch ratio along its length and a low stretch ratio across its width. The tape 100 may be cut so that its width extends transversely across the roll 900, to provide the tape 100 with a high stretch ratio in its widthwise direction. The shapes labelled as tape 100 in FIG. 9 are intended to illustrate the orientation of the tape 100 with respect to the roll 1100 as the tapes 100 are cut from the roll.

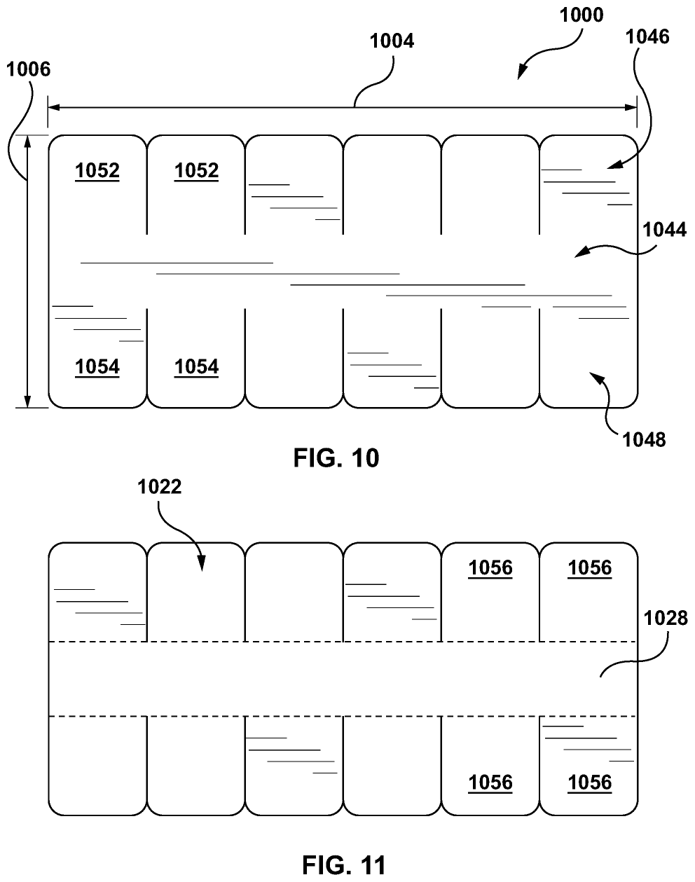

Referring now to FIGS. 10 and 11, an alternative tape 1000 is shown. The tape 1000 is similar to the tape 100 of FIGS. 1 to 8, and similar features to the tape 100 will be referred to with similar reference numerals, incremented by 900.

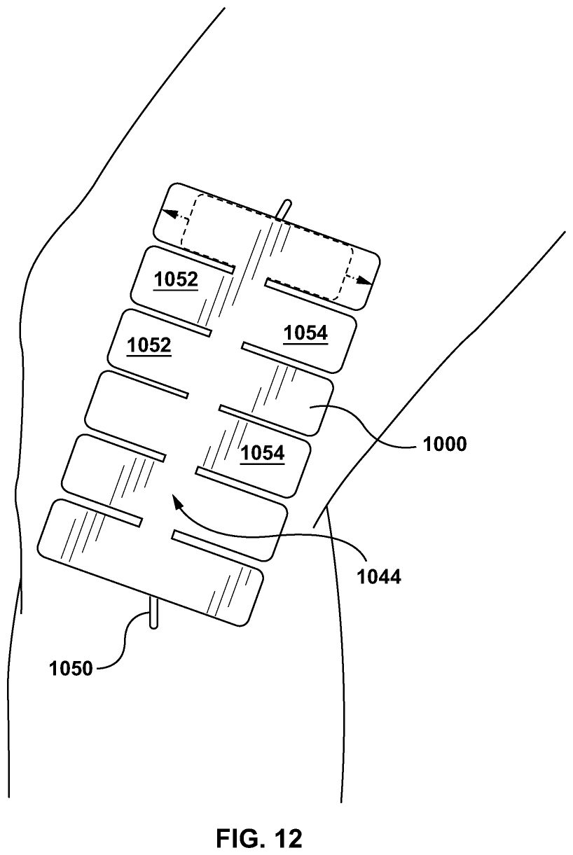

Similarly to the tape 100, the tape 1000 has a length 1004 and a width 1006, and a width stretch ratio that is greater than the length stretch ratio. The tape 1000 further has a central portion 1044 that extends longitudinally, a first side portion 1046 on a first side of the central portion 1044 and a second side portion 1048 on a second side of the central portion 1044. The first side portion 1046 includes a plurality of fingers 1052 (only two of which are labelled in FIG. 10) that extend outwardly from the central portion 1044, and the second side portion 1048 includes a second plurality of fingers 1054 (only two of which are labelled in FIG. 10) that extend outwardly from the central portion 1044. The fingers 1052, 1054 are stretchable away from the central portion 1044, as shown in FIG. 12.

Referring to FIG. 11, a release liner 1022 is on the second face of the tape. The release liner 1022 is perforated along a boundary between the fingers 1052, 1054 and the central portion 1044. This can allow for the central portion 1028 of the release liner 1022 be removed separately from the portions of the release liner on the fingers 1052, 1054, by tearing along the perforations. The portions of the release liner on the fingers 1052, 1054 may be referred to herein as ‘finger release liners 1056’)

Referring to FIG. 12, in use, the release liner 1022 can be torn along the perforations, to remove the central portion 1028 of the release liner. The central portion 1044 of the tape 1000 can then be adhered directly to the scar 1050. For each finger 1052, 1054, the finger release liner 1056 can be removed, and the finger 1052, 1054 can be stretched away from the central portion 1044, and adhered to the skin on the side of the scar 150. The stretchable nature of the fingers is shown schematically in dotted line in FIG. 12. Due to the elastic nature of the tape, the opposed sides of the scar are pulled together by the fingers, along the length of the scar.

1. A method for scar care, comprising:
a. providing a strip of woven material having i) a first face and an opposed second face, ii) a length and a width, wherein the width is shorter than the length, iii) a length stretch ratio along the length and a width stretch ratio along the width, wherein the width stretch ratio is greater than the length stretch ratio, and iv) a first side portion and a second side portion spaced widthwise from the first side portion; wherein an adhesive is on the first face in a discontinuous pattern;
b. adhering the first side portion of the tape to a patient's skin on a first side of a scar;
c. stretching the tape widthwise; and
d. adhering the second side portion of the tape to the patient's skin on a second side of the scar.

2. The method of claim 1, wherein the tape further comprises a central portion between the first side portion and the second side portion, and the first side portion comprises a first plurality of fingers extending outwardly from the central portion, and the second side portion comprises a second plurality of fingers extending outwardly from the first side portion.

3. The method of claim 2, wherein the method further comprises e) adhering the central portion to the scar

4. The method of claim 3, wherein step e) is carried out before step c), and step c) is carried out before steps b) and d).

5. A tape for scar care, comprising:
a. a strip of woven material having i) a first face and an opposed second face, ii) a length and a width, wherein the width is shorter than the length; iii) a length stretch ratio along the length and a width stretch ratio along the width, wherein the width stretch ratio is greater than the length stretch ratio; and iv) a longitudinally extending central portion, a first side portion on a first side of the central portion, wherein the first side portion comprises a first plurality of fingers extending outwardly from the central portion, and a second side portion on a second side of the central portion, wherein the second side portion comprises a second plurality of fingers extending outwardly from the central portion; and
b. an adhesive on the first face in a discontinuous pattern;

6. The tape of claim 5, further comprising a release liner on the second face of the tape.

7. The tape of claim 6, wherein the release liner is perforated along a boundary between the central portion and the first plurality of fingers and the along a boundary between the central portion and the second plurality of fingers.

8. The tape of claim 6, wherein the tape is configured to provide compression to a scar, leading to pressure delivered continuously to the scar.
